(19) United States
(12) Patent Application Publication
(10) Pub. No.: US 2014/0088552 A1
(43) Pub. Date: Mar. 27, 2014

(54) Intravitreal Injection Device

(71) Applicants: Chetan Rasiklal Soni, Springfield, MO (US); Nathan Hesemann, St. Louis, MO (US)

(72) Inventors: Chetan Rasiklal Soni, Springfield, MO (US); Nathan Hesemann, St. Louis, MO (US)

(73) Assignee: THE CURATORS OF THE UNIVERSITY OF MISSOURI, Columbia, MO (US)

(21) Appl. No.: 14/007,121

(22) PCT Filed: Mar. 26, 2012
(86) PCT No.: PCT/US12/30548

§ 371 Date: Dec. 10, 2013

Related U.S. Application Data
(60) Provisional application No. 61/465,882, filed on Mar. 25, 2011.

(51) Int. Cl. A61F 9/00

(52) U.S. Cl. CPC A61F 9/0026
USPC 604/506; 604/117

(57) ABSTRACT

The present disclosure provides a new and improved intravitreal injection device, which is capable of locating the injection site with precision and reproducibility, ensuring the smooth delivery of medications or other treatments, and reducing potential need contaminations. In various embodiments, the device comprises a head unit and a hollow stem extending orthogonally from a bottom of the head unit. The device additionally includes a locating foot slidingly disposed on a distal end of the stem and a biasing device structured to bias the locating foot toward an extended position.

### CROSS-REFERENCE TO RELATED APPLICATIONS

This application claims the benefit of U.S. Provisional Application No. 61/465,882, filed on Mar. 25, 2011. The disclosure of the above application is incorporated herein by reference in its entirety.

### FIELD

The present disclosure relates to a medical device for intravitreal injections, more specifically, to an intravitreal injection device that provides precise and reproducible delivery of medications or other treatments to a target injection site.

### BACKGROUND

Intravitreal injections are frequently used for primarily three purposes: the most common use involves injecting medications, or other treatments, to treat age-related macular degeneration; the second use involves injecting an expansible gas for management of retinal detachment; and the least common use involves injection of antibiotics in case of infection of the inner contents of the eye. Currently the success or failure of an intravitreal injection relies on the experience of the physician performing the procedure. The injection needle has to be placed at a precise location and directed at a correct angle to avoid injury to the internal structures of the eye, which creates a significant learning curve to a novice while performing the procedure.

### SUMMARY

The present disclosure provides an intravitreal injection device that is structured and operable to locate an injection site with precision and reproducibility and to reduce potential needle contaminations. Accordingly, the present intravitreal injection device ensures the smooth, reliable and accurate delivery of intravitreal medications, or other treatments, and significantly reduces the inherent risks commonly associated with known intravitreal injection methods. In various embodiments, the device comprises a head unit and a hollow stem extending orthogonally from a bottom of the head unit. The device additionally includes a locating foot slidingly disposed on a distal end of the stem and a biasing device structured to bias the locating foot toward an extended position.

### BRIEF DESCRIPTION OF DRAWINGS

The drawings described herein are for illustration purposes only and are not intended to limit the scope of the present teachings in any way.

FIG. 1(a) is a schematic of the device for accurately locating an intravitreal injection, in accordance with various embodiments of the present disclosure.

FIG. 1(b) is a bottom view of the device shown in FIG. 1(a), in accordance with various embodiments of the present disclosure.

FIG. 1(c) is a schematic of the device shown in FIG. 1(a) having a locating foot of the device in a depressed position, in accordance with various embodiments of the present disclosure.

FIG. 1(d) is a top view of the device shown in FIG. 1(a), in accordance with various embodiments of the present disclosure.

FIG. 2(a) is an isometric top view of the device shown in FIG. 1(a), in accordance with various embodiments of the present disclosure.

FIG. 2(b) is a cross-sectional view of the device shown in FIG. 1(a), in accordance with various embodiments of the present disclosure.

FIG. 3(a) is an isometric view of the device shown in FIG. 1(a) removably connected to a hypodermic needle, in accordance with various embodiments of the present disclosure.

FIG. 3(b) is a side view of the device shown in FIG. 1(a) removably connected to a hypodermic needle that is connected to a syringe, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

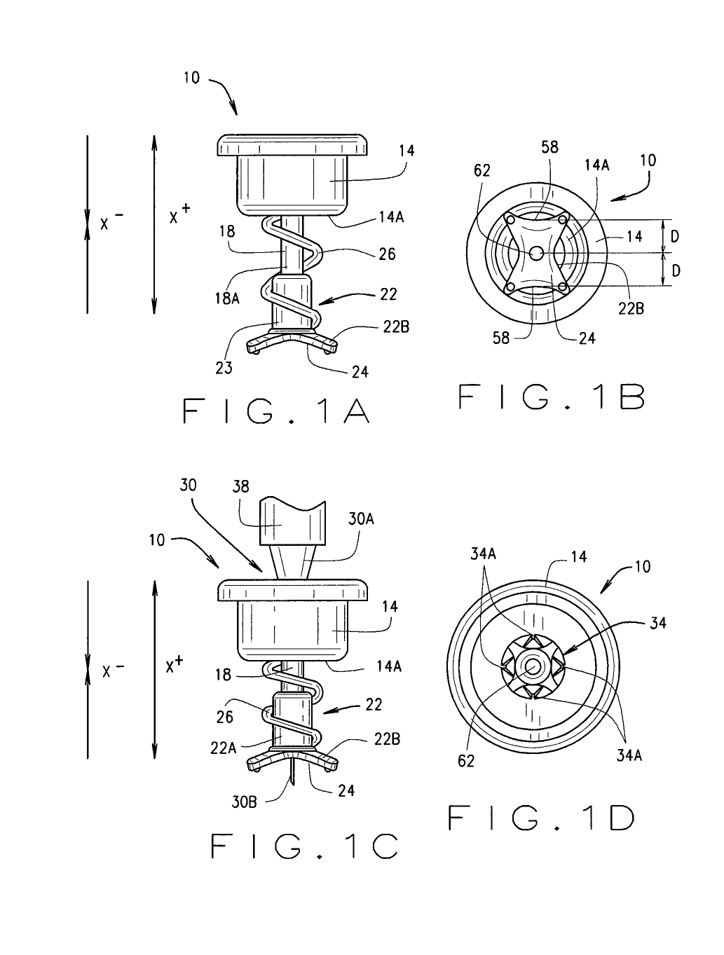

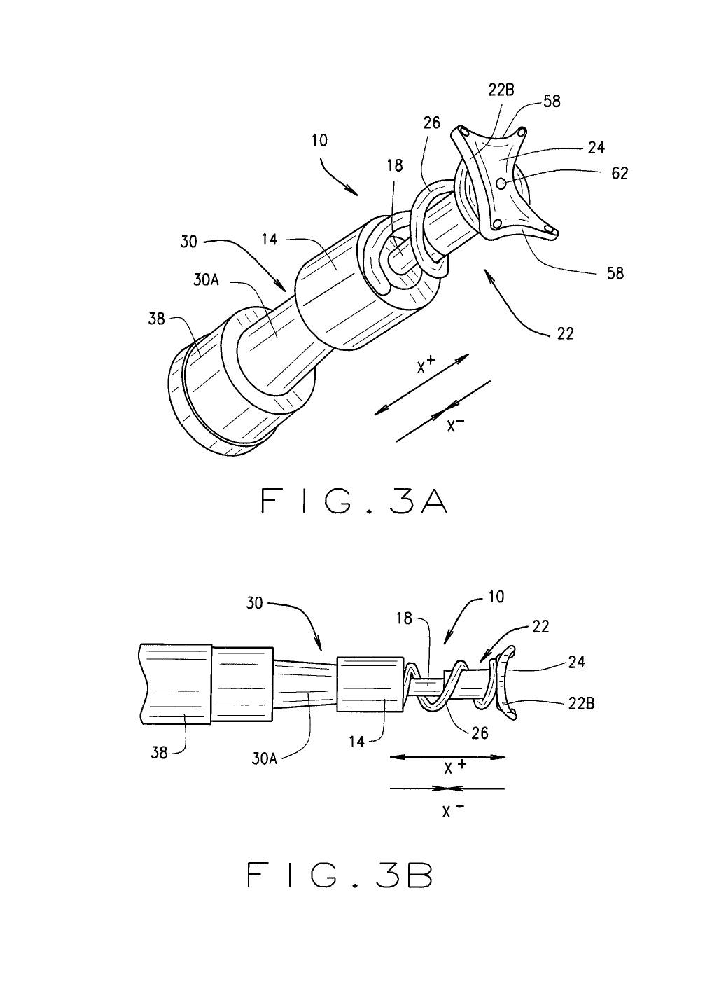

Referring to FIGS. 1(a) through 3(b), in accordance with various embodiments, the present disclosure provides an injection locating device 10 structured and operable to accurately locate an intravitreal injection. The device 10 includes a head unit 14, a hollow stem 18 extending orthogonally from a bottom 14A of the head unit 14, a locating foot 22 slidingly disposed on a distal end 18A of the stem 18, and a biasing device 26 structured to bias the locating foot 22 toward an extended position, shown in FIG. 1(a). Generally, the device 10 is connectable to a hypodermic needle 30, as shown in FIGS. 1(c), 3(a) and 3(b), or more particularly to a connector cap 30A of the hypodermic needle 30, and is structured and operable to accurately locate an injection, to be delivered via a shaft 30B of the hypodermic needle 30, i.e., the needle portion of the hypodermic needle 30, at the pars plana of a human eye and into the mid-vitreous cavity where there are no critical structures. Hence, intravitreal injections delivered using the device 10, as described further below, will not cause damage to the eye. As used herein, the pars plana will be understood to mean the part of the eye that is approximately 3.5 mm to 5.0 mm from the limbus of the eye.

The head unit 14 is formed to include a needle retention fixture 34 that is structured and operable to removably retain the hypodermic needle 30 therein. In various embodiments, the needle retention fixture is sized and structured to receive and removably retain the connector cap 30A of the hypodermic needle 30, wherein the connector cap 30A is designed to connect the hypodermic needle 30 to a syringe 38 or other suitable medication or treatment dispensing device. It is envisioned that connector cap 30A can be any known or unknown hypodermic needle connector cap, such as a well-known Luer-Lok® type connector cap. In various embodiments, the needle retention fixture 34 is a reservoir formed within a center of the head unit 14 having raised ridges 34A disposed along the wall of the reservoir. The raised ridges 34A are structured and operable to frictionally engage the hypodermic needle connector cap 30A, thereby firmly retaining the device 10 on the hypodermic needle 30.

It is envisioned that the needle retention fixture 34 can comprise any other structure(s) or device(s) suitably structured and operable to firmly retain the hypodermic needle connector cap 30A within the head unit 14. For example, in various implementations, the retention fixture 34 can comprise a reservoir formed within a center of the head unit 14 having one or more annular or spiraled protuberances formed along the wall of the reservoir. Or, alternatively, the retention fixture 34 can comprise a reservoir formed within a center of the head unit 14 having a latching or clasping mechanism disposed on the top surface of the head unit 14, whereby the latching or clasping mechanism is operable to latch or clasp the hypodermic needle cap 30A within the reservoir.

Furthermore, it is envisioned that in other embodiments the needle retention fixture 34 can be structured and operable to be removably retained on the hypodermic needle 30 by frictionally, or otherwise, engaging the shaft 30B of the hypodermic needle 30.

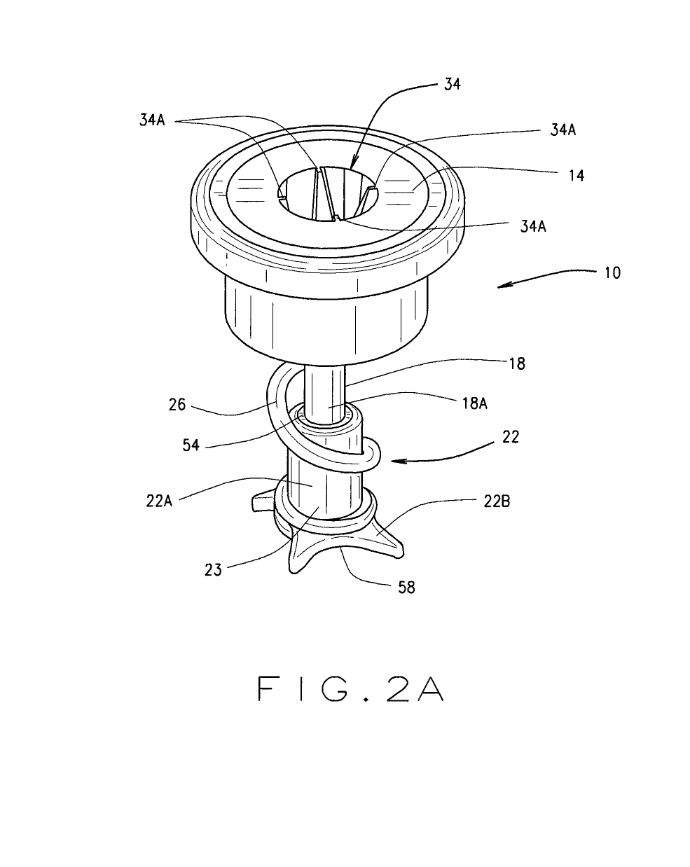

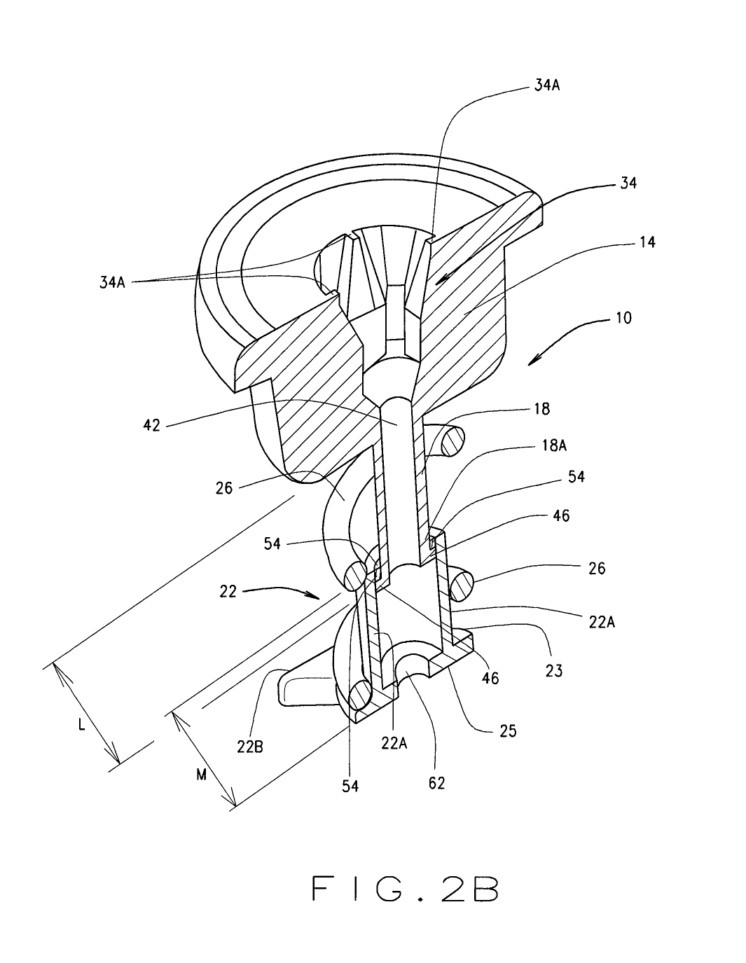

As described above, the stem 18 is hollow and extends orthogonally from the bottom 14A of the head unit 14. More particularly, the stem 18 includes a longitudinal lumen 42 (shown in FIG. 2(b)) that extends the entire length of the stem 18 and is fluidly connected to the needle retention fixture 34, i.e., the lumen 42 opens into the needle retention fixture 34. Therefore, when the connector cap 30A of the hypodermic needle 30 is retained within the needle retention fixture 34 the shaft 30B of the hypodermic needle 30 will extend into the lumen 42 and be protected by the stem 18 and locating foot 22 from contamination by the ambient environment or inadvertent contact with the patient's eyelids, eyelashes, etc. As shown in FIG. 2(b), in various implementations, the stem 18 additionally includes a terminus check 46 disposed or formed at the distal end 18A of the stem 18. The terminus check 46 is structured and operable to retain the locating foot 22 on the distal end 18A, as described further below.

In various embodiments, the locating foot 22 is structured to include a hollow neck 22A and a sole plate 22B disposed or formed at a distal end 23 of the neck 22A. In various implementations, a bottom surface 24 of the sole plate 22B has a concave shape that will provide a conforming contact interface with the globe of a human eye when the locating foot 22 is placed in contact with the globe, as described below. Particularly, in various implementations, the concave bottom surface 24 has a radius of curvature that is substantially equivalent to the radius of curvature of the globe of a human eye, for example 11.0 mm to 13.0, e.g., 12.0 mm. The neck 22A includes a longitudinal bore 50 (shown in FIG. 2(b)) that extends the length of the neck 22A. The bore 50 is sized to receive the stem 18 such that the locating foot 22 can longitudinally slide along the distal portion 18A of the stem 18. Additionally, the locating foot 22 includes an annular stop 54 that is integrally formed or disposed within the bore 50. The annular stop 54 is structured and operable to engage the terminus check 46 of the stem 18 to prevent the locating foot 22 from sliding off the distal end 18A of the stem 18. More particularly, the biasing device 26 is structured and operable to exert a force on the locating foot 22 that will force the locating foot 22 in the X⁺ direction, away from the head unit 14. Therefore, when the device 10 is in a static state, i.e., not having an opposing force applied to the device 10 in the X⁻ direction, the locating foot 22 will be forced to an ‘Extended’ position by the biasing device 26, as shown in FIGS. 1(a), 2(a and b) and 3(a and b). When in the ‘Extended’ position, the annular stop 54 of the locating foot 22 is in contact with terminus check 46 of the stem 18 and the locating foot 22 is prevented from being pushed off the stem 18 by the biasing device 26.

As described above, when the hypodermic needle 30 is retained within the needle retention fixture 34 the shaft 30B of the hypodermic needle 30 will extend into the stem lumen 42. Furthermore, the stem 18 is structured to have a particular length L (shown in FIG. 2(b)) such that a desired portion of the hypodermic needle shaft 30B, e.g., 5/16 to 13/16 of an inch or 7.9 mm to 20.6 mm, will extend beyond the distal end 18A of the stem 18 and protrude into the bore 50 of the locating foot neck 22A. Additionally, the locating foot neck 22A is structured to have a particular length M (shown in FIG. 2(b)) such that the portion of the hypodermic needle shaft 30B that extends beyond the stem distal end 18A is entirely disposed within the bore 50 and protected from contamination by the locating foot 22 when the locating foot 22 is in the ‘Extended’ position.

Referring particularly to FIGS. 1(b) and 2(b), the locating foot sole plate 22B includes at least one curved edge 58. As exemplarily illustrated in FIG. 1(b), in various embodiments, the sole plate 22B can have opposing curved edges 58. Each curved edge 58 has a radius of curvature substantially equal to the radius of curvature of the limbus of a human eye, for example 5.0-6.5 mm, e.g., 5.5 mm. The locating foot sole plate 22B additionally includes an egress aperture 62 that extends through a center of the sole plate 22B and is fluidly connected to the bore 50 within the neck 22A. The egress aperture 62 is sized to allow the shaft 30B of a hypodermic needle 30 to pass therethrough, as shown in FIG. 1(c).

Importantly, the egress aperture 62 is located at a specific target zone distance D from a center point of each curved edge 58, e.g., 3.5 mm to 4.0 mm from the center point of the curved edge 58. Therefore, an operator, e.g., a physician or ophthalmologist, can place the bottom surface 24 of sole plate 22B in contact with the globe of a human eye having the, or one of the, curved edge(s) 58 aligned with the limbus such that the egress aperture 62 is accurately and reproducibly located the target zone distance D from the limbus, e.g., 3.5 mm to 4.0 mm, where medication, or other treatment, can be delivered into the mid-vitreous cavity, via the hypodermic needle 30, without causing damage to the eye. It is important to understand that the target zone distance D is specifically calculated/determined to be a distance equal to a radial distance from the limbus, i.e., a substantially orthogonal distance from the limbus along the surface of the globe of the eye, i.e., the conjunctiva and sclera, that is known to identify a specific location on the globe where an intravitreal injection can occur in the mid-vitreous cavity without penetrating or damaging any critical structures of the eye, and hence, without damaging the eye.

Referring now to FIGS. 1(a) and 1(b), the biasing device 26 is structured to have a specific biasing force that will maintain the locating foot 22 in the ‘Extended’ position when the device 10 is in a static state, but can be easily overcome by applying a light force in the X⁻ direction to the device 10. More specifically, the biasing force of the biasing device 26 is calibrated to retain the locating foot 22 in the ‘Extended’ position when the sole plate 22B is not in contact with the human eye, but can be easily overcome by an opposing force in the X⁻ direction applied by the operator, e.g., physician or ophthalmologist, to the head unit 14 or the syringe 38 (or other medication or treatment dispensing device having the hypodermic needle 30 connected thereto) when the sole plate 22B is aligned with the limbus and placed in contact with the globe of the eye.

Particularly, when the injection locating device 10 is engaged with the hypodermic needle 30, as described above, (the hypodermic needle 30 being connected to a syringe 38 or other medication or treatment dispensing device) and the operator, aligns an edge 58 with the limbus and places the sole plate 22B in contact with the eye, the operator can apply a force to the syringe 38 (or other medication or treatment dispensing device) and/or head unit 14 downward toward the eye. This downward force will overcome the biasing force of the biasing device 26 in the X⁺ direction and cause the neck 22A of the locating foot 22 to slide along the distal end 18A of the stem 18 toward the head unit 14 to a depressed or ‘Injection’ position, shown in FIG. 1(c), wherein the distal end 18A of the stem 18 abuts and contacts an upper surface 25 of the sole plate 22B within the bore 50 of locating foot neck 22A. The biasing force of the biasing device 26 is calibrated such that the force applied by the operator to overcome the biasing force will not damage the anatomy of the eye.

Consequently, this downward force will cause the hypodermic needle shaft 30B to protrude through the egress aperture 62 in the sole plate 22B and penetrate the eye at the target zone distance D from the limbus such that the needle shaft 30B will enter the mid-vitreous cavity of the eye. Thereafter, the operator can inject a medication or treatment disposed in the syringe 38 (or other medication or treatment dispensing device) into the eye via the appropriate operation of the syringe 38 (or other medication or treatment dispensing device).

As described above, the neck 22A of the locating foot 22 is structured to have a particular length M. Importantly, the length M is calibrated such that when the locating foot 22 is depressed to the ‘Injection’ position, as described above, only a desired portion of the hypodermic needle shaft 30B, e.g., ¼ to ¾ of an inch or 6.3 mm to 19.0 mm, will extend beyond the bottom surface 24 of the sole plate 22B. More specifically, the length M of the neck 22A is calibrated to gauge the depth of penetration of the needle shaft 30B into the mid-vitreous cavity of the eye, e.g., ¼ to ¾ of an inch or 6.3 mm to 19.0 mm.

The biasing device 26 can be any device, structure or mechanism structured to have a specific biasing force in the X⁺ direction that will maintain the locating foot 22 in the ‘Extended’ position when the device 10 is in a static state, but can be easily overcome by applying a light force in the X⁻ direction to the device 10, as described above. For example, in various embodiments, the biasing device 26 can be a light weight coil spring, as shown throughout the figures. Alternatively, the biasing device 26 can be one or more leaf springs, a flexible and resilient sleeve or gasket disposed over the stem 18, or any other suitable actuator or spring-like device, structure or mechanism.

Referring particularly to FIGS. 1(b) and 3(a), in various embodiments the locating foot 22 can include a plurality of stabilizing bosses 66 extending from the bottom surface 24 of the sole plate 22B. The stabilizing bosses are structured and operable to maintain the position of the sole plate 22B on the globe of the eye once the sole plate 22B has been positioned and placed on the eye, as described above. Particularly, when the operator applies the downward force to the syringe 38 (or other medication or treatment dispensing device) and/or head unit 14, the bosses 66 will protrude slightly into the surface of the eye, i.e., the conjunctiva and sclera, thereby stabilizing the locating foot 22, and assisting in keeping the sole plate 22B aligned with the limbus and the egress aperture 62 located the target zone distance D from the limbus.

While the disclosure has been described in connection with specific embodiments thereof, it will be understood that the device is capable of further modifications. This patent application is intended to cover any variations, uses, or adaptations of the disclosure following, in general, the principles of the disclosure and including such departures from the present disclosure as come within known or customary practice within the art to which the disclosure pertains and as may be applied to the essential features herein.

1. A device for accurately locating an intravitreal injection, said device comprising:
a head unit;
a hollow stem extending orthogonally from a bottom of the head unit;
a locating foot slidingly disposed on a distal end of the stem; and
a biasing device structured to bias the locating foot toward an extended position.

2. The device of claim 1, wherein the head unit comprises a needle retention fixture structured to removably retain a connector cap of a hypodermic needle.

3. The device of claim 2, wherein the stem comprises:
a longitudinal lumen extending the length of the stem and fluidly connected to the needle retention fixture such that a shaft of a hypodermic needle having the connector cap retained within the needle retention fixture will extend into the lumen and be protected by the stem; and
a terminus check disposed at the distal end of the stem.

4. The device of claim 3, wherein the locating foot comprises:
a hollow neck; and
a sole plate disposed at a distal end of the neck and having a concave bottom surface with a radius of curvature substantially the same as the radius of curvature of the globe of a human eye.

5. The device of claim 4, wherein the locating foot further comprises:
a longitudinal bore extending the length of the neck and sized to receive the stem such that the locating foot can longitudinally slide along a distal portion of the stem; and
an annular stop disposed within the bore, the annular stop structured and operable to be engageable with the terminus check of the stem to prevent the locating foot from sliding off the distal end of the stem, the extended position of the locating foot being when the annular stop is engaged with the terminus check.

6. The device of claim 5, wherein:
the hollow stem is structured to have a particular length such that a desired portion of the shaft of a hypodermic needle retained within the device, via the head unit needle retention fixture, will extend beyond the distal end of the stem; and
the locating foot neck is structured to have a particular length such that the portion of the hypodermic needle shaft extending beyond the distal end of the stem is entirely disposed and protected within the bore of the locating foot when the locating foot is in the extended position.

7. The device of claim 5, wherein the locating foot further comprises:
at least one curved edge having a radius of curvature substantially equal to the radius of curvature of the limbus of a human eye; and
an egress aperture extending through a center of the sole plate and fluidly connected to the bore within the neck, the egress aperture located at a specific target zone distance from a center point of the curved edge and sized to allow the shaft of a hypodermic needle to pass therethrough, whereby the sole plate can be placed in contact with the globe a human eye having the curved edge aligned with the limbus such that the locating foot egress aperture is located the target zone distance from the limbus.

8. The device of claim 7, wherein the locating foot further comprises a plurality of stabilizing bosses disposed on the bottom surface of the sole plate, the bosses structured and operable to maintain the position of the sole plate on the eye once the curved edge is aligned with the limbus.

9. The device of claim 7, wherein the biasing device is structured to have a biasing force calibrated to:
retain the locating foot in the extended position when the sole plate is not in contact with the human eye, and
easily be overcome by an opposing force applied by an operator to the head unit when the sole plate is placed in contact with the eye such that the locating foot will slide along the stem toward the head unit to an injection position whereby the shaft of a hypodermic needle retained within the device, via the head unit needle retention fixture, will protrude through the egress aperture in the sole plate and penetrate the eye at the target zone distance from the limbus and be disposed within the mid-vitreous cavity, without causing damage to the eye.

10. A device for accurately locating an intravitreal injection, said device comprising:
a head unit comprising a needle retention fixture structured to removably retain a connector cap of a hypodermic needle;
a hollow stem extending orthogonally from a bottom of the head unit;
a locating foot slidingly disposed on a distal end of the stem, the locating foot comprising:
a hollow neck having a longitudinal bore extending the length of the neck and sized to receive the stem such that the locating foot can longitudinally slide along a distal portion of the stem; and
a sole plate disposed at a distal end of the neck, the sole plate comprising:
at least one curved edge having a radius of curvature substantially equal to the radius of curvature of the limbus of a human eye; and
an egress aperture extending through a center of the sole plate and fluidly connected to the bore within the neck, the egress aperture located at a specific target zone distance from a center point of the curved edge and sized to allow the shaft of a hypodermic needle retained in the head unit by the retention fixture to pass therethrough, whereby the sole plate can be placed in contact with the globe of a human eye having the curved edge aligned with the limbus such that the locating foot egress aperture is located the target zone distance from the limbus; and
a biasing device structured to bias the locating foot toward an extended position.

11. The device of claim 10, wherein the stem comprises:
a longitudinal lumen extending the length of the stem and fluidly connected to the needle retention fixture such that a shaft of a hypodermic needle having the connector cap retained within the needle retention fixture will extend into the lumen and be protected by the stem; and
a terminus check disposed at the distal end of the stem.

12. The device of claim 10, wherein the sole plate of the locating foot further comprises a concave bottom surface with a radius of curvature substantially the same as the radius of curvature of the globe of a human eye.

13. The device of claim 11, wherein the locating foot further comprises an annular stop disposed within the bore, the annular stop structured and operable to be engageable with the terminus check of the stem to prevent the locating foot from sliding off the distal end of the stem, the extended position of the locating foot being when the annular stop is engaged with the terminus check.

14. The device of claim 11, wherein:
the hollow stem is structured to have a particular length such that a desired portion of the shaft of a hypodermic needle retained within the device, via the head unit needle retention fixture, will extend beyond the distal end of the stem; and
the locating foot neck is structured to have a particular length such that the portion of the hypodermic needle shaft extending beyond the distal end of the stem is entirely disposed and protected within the bore of the locating foot when the locating foot is in the extended position.

15. The device of claim 10, wherein the locating foot further comprises a plurality of stabilizing bosses disposed on the bottom surface of the sole plate, the bosses structured and operable to maintain the position of the sole plate on the eye once the curved edge is aligned with the limbus.

16. The device of claim 10, wherein the biasing device is structured to have a biasing force calibrated to:
retain the locating foot in the extended position when the sole plate is not in contact with the human eye, and
easily be overcome by an opposing force applied by an operator to the head unit when the sole plate is placed in contact with the eye such that the locating foot will slide along the stem toward the head unit to an injection position whereby the shaft of a hypodermic needle retained within the device, via the head unit needle retention fixture, will protrude through the egress aperture in the sole plate and penetrate the eye at the target zone distance from the limbus, such that the hypodermic needle shaft will be disposed within the mid-vitreous cavity of the eye and will not cause damage to the eye.

17. A method for accurately locating an intravitreal injection, said method comprising:
removably retaining a connector cap of a hypodermic needle within a needle retention fixture of a head unit of an intravitreal injection device such that a shaft of the hypodermic needle is disposed within a longitudinal lumen of a hollow stem extending orthogonally from a bottom of the head unit, the hypodermic needle coupled with a syringe via the connector cap, the hollow stem having a particular length such that a desired portion of the hypodermic needle shaft extends beyond a distal end of the stem and into a bore within a neck of a locating foot of the intravitreal injection device that is slidingly disposed on the distal end of the stem and biased toward an extended position via a biasing device of the intravitreal injection device; and
aligning a curved edge of a sole plate disposed at a distal end of the locating foot neck with the limbus of a human eye and placing a concave bottom surface of the sole plate in contact with the globe of the eye, the sole plate including an egress aperture located at a specific target zone distance from a center point of the curved edge such that when the curved edge is aligned with the limbus and the sole plate is placed in contact with the globe the egress aperture is positioned the target zone distance from the limbus; and
applying a force to the head unit sufficient to overcome the biasing force of the biasing device such that the locating foot will slide along the stem toward the head unit to an injection position whereby the shaft of the hypodermic needle retained within the device will protrude through the egress aperture in the sole plate and penetrate the eye at the target zone distance from the limbus without causing damage to the eye.

18. The method of claim 17 further comprising maintaining the position of the sole plate on the globe of the eye once the curved edge is aligned with the limbus by applying a force to the head unit sufficient to overcome the biasing force and cause a plurality of stabilizing bosses disposed on the concave bottom surface of the sole plate to slightly protrude into the surface of the globe of the eye.
